(19) United States
(12) Patent Application Publication
(10) Pub. No.: US 2022/0395253 A1
(43) Pub. Date: Dec. 15, 2022

(54) Creation of a flexible ultrasound system for real time acquisition of large fields of view

(71) Applicant: The Board of Trustees of the Leland Stanford Junior University, Stanford, CA (US)

(72) Inventors: Katherine W Ferrara, Palo Alto, CA (US); Josquin L Foiret, Mountain View, CA (US); Xiran Cai, Redwood City, CA (US)

(21) Appl. No.: 17/765,257

(22) PCT Filed: Oct. 2, 2020
(86) PCT No.: PCT/US2020/054009

§ 371 Date: Mar. 30, 2022

Related U.S. Application Data
(60) Provisional application No. 62/910,875, filed on Oct. 4, 2019.
(60) Provisional application No. 63/074,813, filed on Sep. 4, 2020.

(51) Int. Cl. A61B 8/00; A61B 8/15

(52) U.S. Cl. CPC A61B 8/4477; A61B 8/15

(57) ABSTRACT

Improved acoustic tomography is provided using an array of transducer modules that surround the target, Each transducer module is a phased array of acoustic transducer elements that provides a steerable plane wave or steerable diverging wave excitation to the target. Tomographic reconstruction of the resulting data sets is substantially less computationally demanding than tomographic reconstruction of conventional acoustic tomography data sets, enabling image frame rates of 10 per second or better. This approach can be combined with dual modality imaging In cases where hardware limitations lead to undesirable gaps between the transducer modules, virtual receiver elements can be defined at locations between the transducer modules. By estimating signals that would be received at locations of the virtual receiver elements, the undesirable effects of these gaps can be reduced.

### FIELD OF THE INVENTION

This invention relates to acoustic imaging.

### BACKGROUND

Ultrasound tomography typically employs circular or conformal transducer arrays to acquire data. In typical transmission tomography, one element transmits the sound wave and the scattered waves are received by other transducer elements facing the transmitter, resulting in large datasets and slow acquisition. This process is then rotated around the array. Computationally intensive iterative methods such as full wave inversion, Born inversion, inverse Radon transform and inverse scattering are then applied to construct the image and estimate parameters. Consequently, reconstruction of one image slice typically requires 10 seconds to several hours resulting in small patient throughput. Further, real-time techniques, including Doppler imaging, elastography and functional imaging have not been developed in the context of ultrasound tomography. Accordingly, it would be an advance in the art to provide improved acoustic tomography.

### SUMMARY

FIG. 1A shows an exemplary embodiment of the invention. This example is an apparatus for performing acoustic tomography. It includes three or more transducer modules 106a,b,c,d,e,f,g,h disposed to surround a target 102. Each transducer module 106a-h includes two or more individually driven acoustic transducer elements, e.g., 107a-d. To reduce clutter on the figure, these references to the transducer elements are not duplicated for transducer modules 106b-h. This apparatus also includes a processor 104 configured to transmit one or more first acoustic signals 108 from a selected transducer module (e.g., 106e) and configured to receive one or more second acoustic signals (110a, 110b, 110c) at two or more of the transducer modules (e.g., 106d, 106e, 106f), wherein the two or more of the transducer modules includes the selected transducer module (106e). Here the one or more first acoustic signals are plane wave excitations or diverging wave excitations provided by the selected transducer module. The processor is further configured to sequentially select each of the transducer modules as the selected transducer module to provide a data set for tomographic reconstruction. For example, each of 106a-h can be the selected transducer module in sequence, and in each case signals can be received by the selected transducer module and by transducer modules adjacent to the selected transducer module. Finally, the processor is configured to provide a first image from the data set for tomographic reconstruction.

Preferably the processor is configured to provide a frame rate for the first image of 10 Hz or more. This capability is a substantial advantage of the present approach, since conventional acoustic tomography can't be done at such high frame rates.

The apparatus can be configured to provide a second image according to a second imaging modality that is co-registered with the first image. FIG. 1B shows an example, where an optical source 112 provides an optical signal 114 to target 102 for photoacoustic imaging using the acoustic receivers 106a-h. Here the two or more of the transducer modules further receive one or more third acoustic signals due to a photoacoustic effect in the target, and the second image is a photoacoustic image determined from the third acoustic signals. Any other imaging modality that is compatible with acoustic tomography can be used as the second imaging modality.

Plane wave excitations can be provided by driving the acoustic transducer elements of the selected transducer module in phase with each other. Here the acoustic transducer elements of the selected transducer module can be driven with a linear phase gradient to provide beam steering.

Diverging wave excitations can be provided by defining a virtual source and driving the acoustic transducer elements of the selected transducer module with phases corresponding to the virtual source. FIG. 1C shows an example. Here 108 is a diverging wave provided by transducer module 106. Relative phases to apply to the elements of transducer module 106 to create diverging wave 108 can be computed by defining a virtual point source 120 and setting phases of transducer elements as if wave fronts 124 from virtual source 102 were incident on transducer module 106 from behind. This approach has the benefit of readily lending itself to beam steering of the diverging wave excitation. FIG. 1D shows an example. Here virtual source 120′ is laterally displaced relative to transducer module 106, thereby steering diverging wave excitation 108′ as shown. Phases of elements of the transducer module set according to wave fronts 126 will provide this result. It is also possible to adjust the divergence of the diverging wave excitation by positioning the virtual source close to the transducer module (high divergence) or far away from the transducer module (low divergence).

Preferably the two or more of the transducer modules does not include all of the transducer modules. This can desirably prevent direct transmission from the selected transducer module to the two or more of the transducer modules.

The data set for tomographic reconstruction can be a reflection tomography data set or a transmission tomography data set. The first image can be a B-mode image or an attenuation image.

Preferably the processor is configured to apply a coherence factor correction to the data set for tomographic reconstruction.

The processor can be configured to determine a speed of sound correction by estimating a target speed of sound in the target and an ambient speed of sound in a medium surrounding the target and between the target and the three or more transducer modules. In some cases, this medium is water.

The processor can be configured to reduce an effect of physical gaps between the transducer modules by defining two or more virtual acoustic elements (e.g., virtual elements 130a and 130b) at locations between the transducer modules. In this example, virtual elements 130a are between transducer modules 106h and 106a, and virtual elements 130b are between transducer modules 106h and 106a. As described in section C below, by estimating received acoustic signals at locations of the virtual acoustic elements it is possible to reduce the effect of the physical gaps between the transducer modules on imaging performance.

### BRIEF DESCRIPTION OF THE DRAWINGS

FIGS. 1A-B show two exemplary embodiments of the invention.

FIGS. 1C-D show steering a diverging beam my moving a virtual source.

FIG. 1E shows virtual sensor elements for reducing an effect of gaps between transducer modules.

FIG. 1F shows the main acquisition sequence for the work of section A.

FIGS. 1G-J show simulated B-mode images for two acquisition sequences and with and without a coherence factor correction.

FIGS. 2A-E relate to B-mode images obtained for two acquisition sequences and with and without a coherence factor correction.

FIGS. 3A-H show the effect of providing a speed of sound correction.

FIGS. 4A-H are rat abdomen images compared for various acquisition sequences.

FIGS. 5A-L demonstrate small animal imaging.

FIGS. 6A-E demonstrate in vivo human hand imaging.

FIGS. 7A-F demonstrate attenuation imaging combined with B-mode imaging.

FIGS. 8A-K show simulation results for a more realistic transducer configuration (having gaps).

FIGS. 9A-F show further simulation results for the configuration of FIG. 8A.

FIGS. 10A-B show imaging results using a metal wire phantom.

FIGS. 11A-B show a comparison of (1 Tx/1 Rx) acquisition to (1 Tx/3 Rx) acquisition.

FIGS. 12A-C show a comparison of (1 Tx/1 Rx) acquisition to 8-view acquisition for human wrist imaging.

FIGS. 13A-F relate to dual mode imaging.

FIGS. 14A-1, 14A-2, 14A-8, 14B and 14C relate to dual mode imaging.

FIGS. 15A-B show the effect of gap compensation on the point spread function.

FIGS. 16A-B relate to gap compensation for imaging a phantom.

FIGS. 17A-D relate to gap compensation for in vivo imaging.

### DETAILED DESCRIPTION

Section A describes in detail an example of improved acoustic tomography according to the above described principles. Section B relates to dual-modality imaging. Section C relates to compensating for gaps between the transducer modules.

### A) Improved Acoustic Tomography

### A1) Introduction

Ultrasound (US) imaging is used throughout medicine; however, operator-dependent acquisition and poor spatial resolution have limited the utility. Conventional ultrasound imaging uses a small linear or 2D matrix probe to transmit (Tx) and receive (Rx) ultrasound signals. B-mode images are typically then reconstructed using reflected signals to provide a representation of the interrogated region. Typical limitations of conventional ultrasound imaging include limited field of view, limited penetration compared to the size of the imaged object and diffraction-limited resolution. Tomography, defined as a technique for displaying a representation of a cross section through a human body, facilitates high resolution (lambda/2) imaging by effectively rotating the US point spread function (PSF) and increasing the aperture to limit the effect of diffraction. Tomographic imaging has the potential to create an operator-independent acquisition protocol.

Ultrasound tomography typically employs circular or conformal transducer arrays to acquire data. In typical transmission tomography, one element transmits the sound wave and the scattered waves are received by other transducer elements facing the transmitter, resulting in large datasets and slow acquisition. This process is then rotated around the array. Computationally intensive iterative methods such as full wave inversion, Born inversion, inverse Radon transform and inverse scattering are then applied to construct the image and estimate parameters. Consequently, reconstruction of one image slice requires 10 seconds to several hours resulting in small patient throughput. Further, real-time techniques, including Doppler imaging, elastography and functional imaging have not been developed in the context of ultrasound tomography. Transmission tomography has been shown to facilitate tissue characterization through estimation of the speed-of-sound (SOS) and attenuation.

The recent development of high channel count ultrafast US systems offers the opportunity to capture images at a high frame rate using plane waves or diverging waves to insonify a large field-of-view. These systems have been leveraged to create vector flow imaging, super resolution imaging and functional brain imaging.

While tomographic systems have been developed in the past, here, we sought to develop a system for real-time quantitative reflection tomography. In reflection tomography, the maximum propagation path length is typically reduced, and the center frequency can be increased as compared with transmission tomography. While transmission tomography cannot yet be successfully applied to image body regions containing bone, we demonstrate that reflection tomography can produce high-quality cross-sectional images of limbs (surrounding longitudinal bones). We use plane wave acquisition to achieve a high-volume acquisition rate, facilitating spatial compounding and further increasing image quality. By combining tomographic acquisition with coherence processing and localized speed of sound correction, high resolution images are obtained. In addition, for the first time, we assess attenuation estimation using tomographic plane wave acquisition. By combining images obtained from multiple acquisition directions, regions of locally enhanced attenuation are quickly recognized, and the attenuation coefficient estimated. In summary, we exploit ultrafast tomographic acquisition to achieve nearly isotropic in-plane resolution (˜150 microns at 5 MHz) and reduce the clutter floor, thus improving image contrast in studies of phantoms, small animals and human volunteers.

### A2) Results

### A2a) Image Quality is Enhanced By the Tomographic Reconstruction

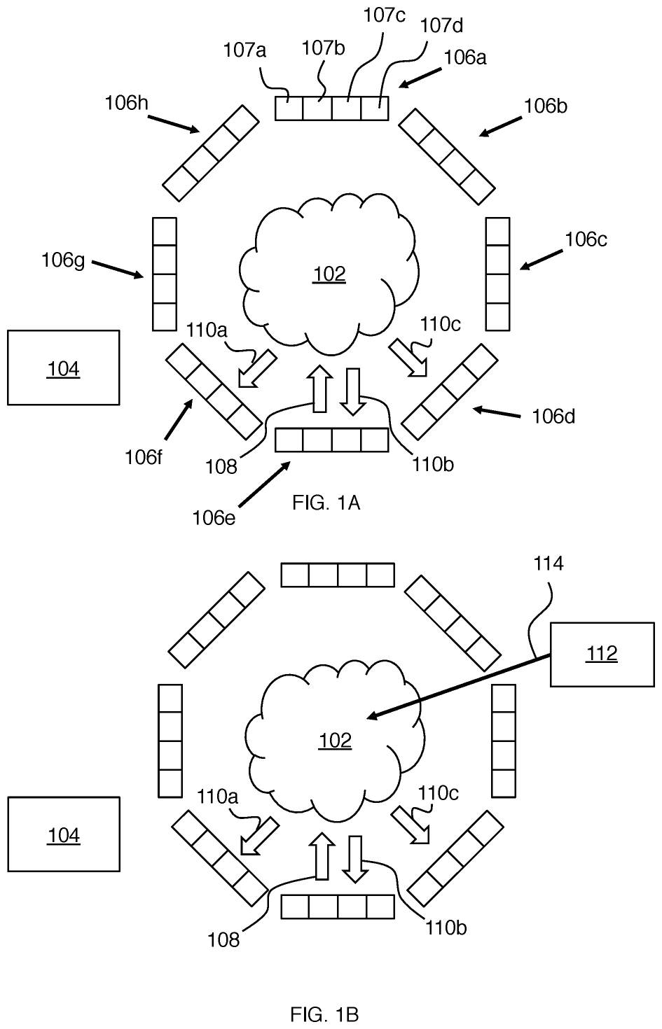

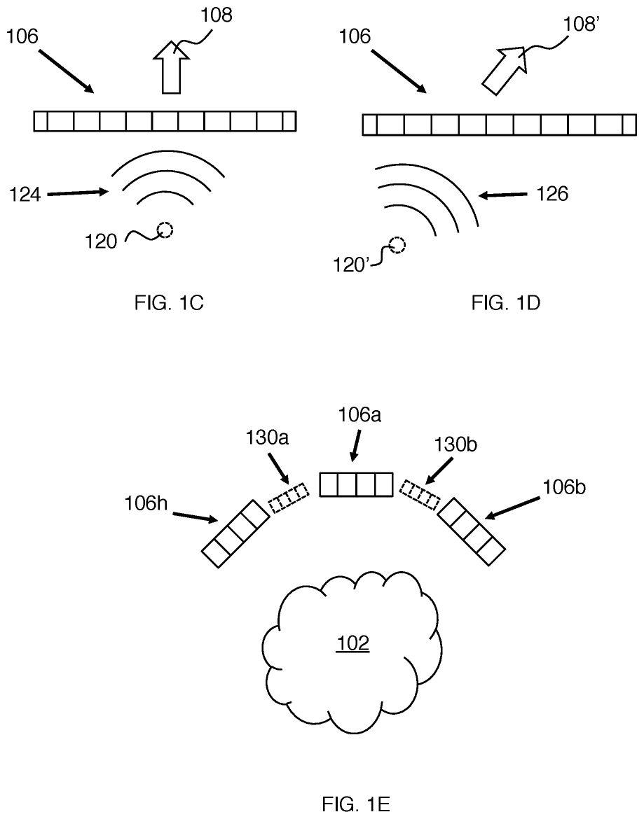

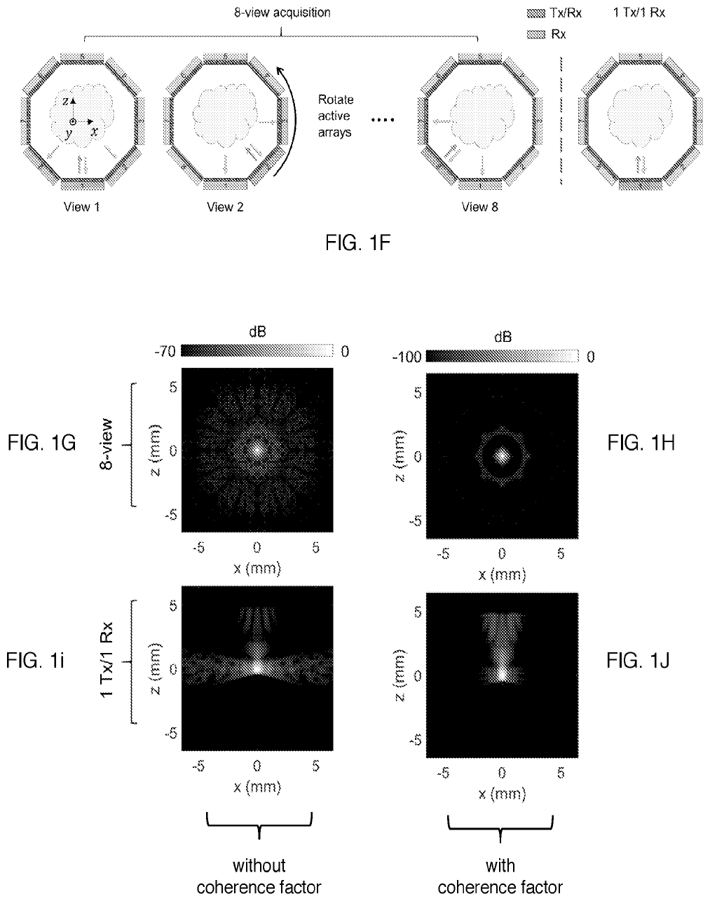

FIG. 1F shows the tomographic acquisition scheme. Here the center array of a group of 3 arrays transmits (Tx) and echoes are received (Rx) by all three active arrays. The active arrays are rotated to acquire data from views 1 to 8 which are then coherently compounded to form the final tomographic image. This acquisition scheme is termed as 8-view acquisition. For comparison, acquisition with a single array activated on transmission and reception is also shown as this is the typical method used for ultrasound acquisition (1 Tx/1 Rx, right side of FIG. 1F). The lateral and axial directions with respect to array 1 are denoted as x- and z-directions in the reconstructed images. For 8-view acquisition, FIGS. 1G and 1H show simulated B-mode images (of a point target located at the center of the arrays of the PSF with or without coherence factor weighting as indicated. The arrays were positioned along a 9.2 cm diameter circle, i.e. the distance between array 1 and 5 was 9.2 cm. For 1 Tx/1 Rx acquisition, FIGS. 1i and 1J show simulated B-mode images of a point target located at the center with or without coherence factor weighting as indicated. For the 1 Tx/1 Rx acquisition, array 1 was activated for transmission (24 plane waves between −13° and) 13° and reception. For the 8-view acquisition, 3 plane waves (−13° to 13°) were transmitted for one view and the active arrays were rotated as described above, then the data from all 8 views were compounded.

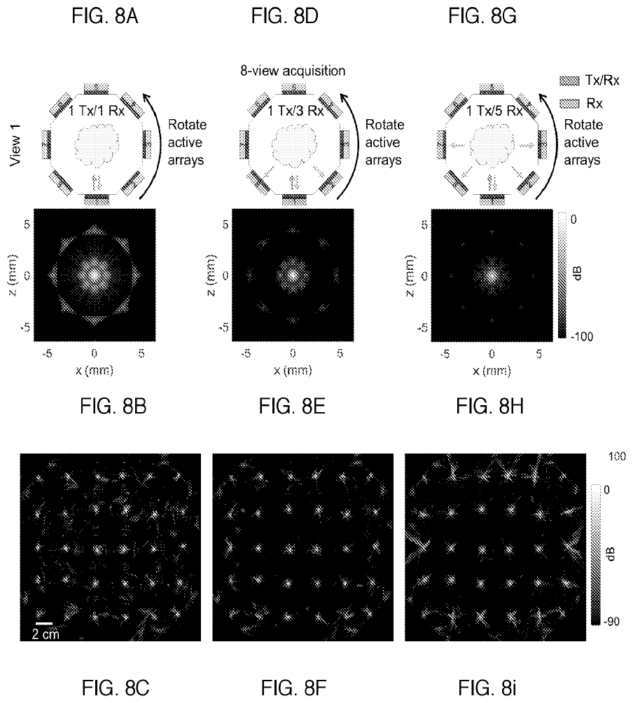

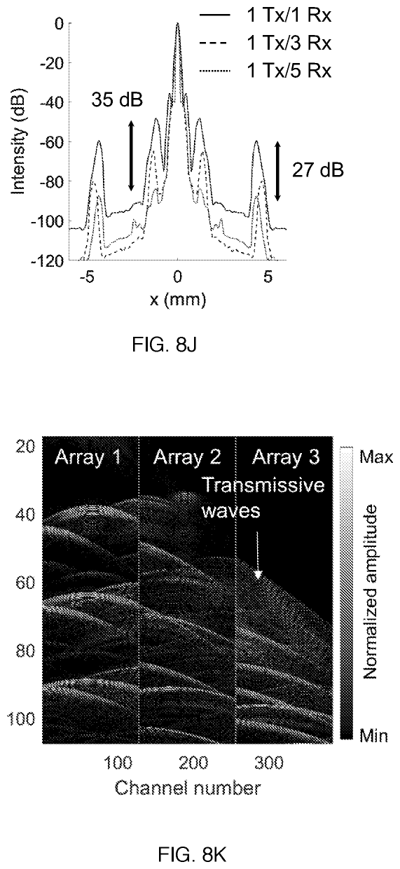

We first evaluated the point spread function (PSF) for the tomographic reconstruction resulting from plane wave insonation with 1 array transmitting for each view and 1, 3 and 5 arrays used on reception, respectively (FIGS. 8A-8i). The active array subset was rotated to sample the full aperture (see an example in FIG. 1F for 8-view acquisition with 1 Tx/3 Rx for each view). For an 8-view acquisition, the spatial resolution was isotropic and improved from 0.20 mm to 0.15 mm using 5 arrays in reception for each view compared to using 1 array in reception, and the side and grating lobes were suppressed by 35 and 27 dB, respectively (FIG. 8J). Experimental data further showed that using more arrays in reception would effectively suppress the grating lobes (FIGS. 8C, 8F, 8i). However, with 5 arrays in reception, the arrays at the two extremities received transmitted waves from the Tx array directly (FIGS. 8i, 8K). Therefore, reception of 3 arrays and 8 views were applied for our tomographic acquisitions.

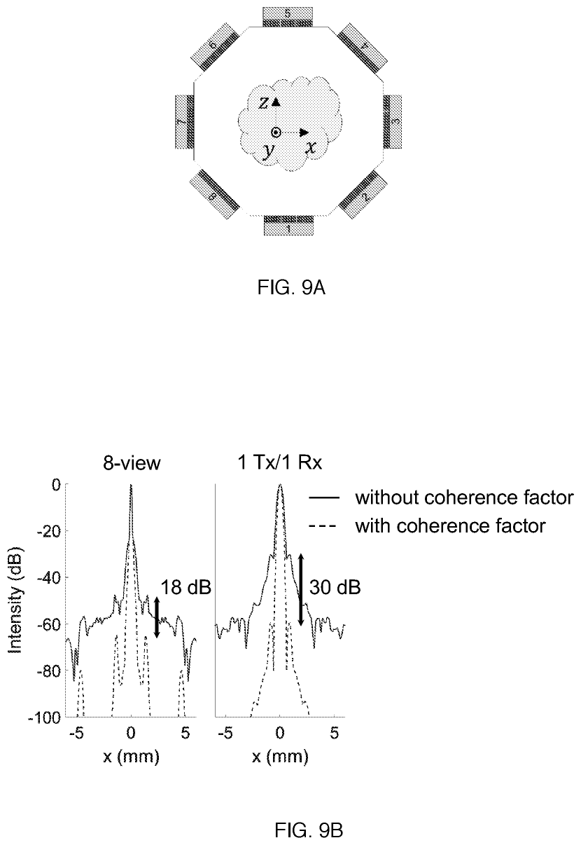

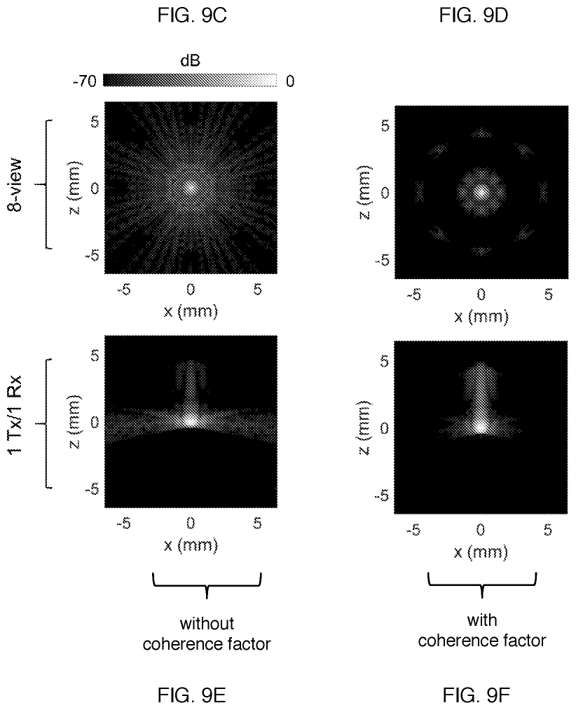

We then further compared the PSF determined both with and without the application of coherence factor (CF) processing. For an 8-view acquisition, nearly half-wavelength isotropic spatial resolution (0.16 mm) was achieved with or without CF (FIGS. 1G, 1H). Using a single array for transmission and reception (1 Tx/1 Rx), the resolution in the z direction was 0.33 mm with or without CF and the resolution in the x direction was 0.41 and 0.25 mm without and with CF, respectively (FIGS. 1i, 1J). Simulations of the exact geometry (FIG. 9A) implemented in the experimental studies (using commercial arrays that force a gap between neighboring arrays and increasing the aperture size to match the active aperture) retained the −6 dB resolution (0.16 mm) obtained with the fully tomographic acquisition (FIGS. 9C, 9D). However, for the 1 Tx/1 Rx acquisition, the spatial resolution was degraded due to the increased distance to the target (FIG. 9E, 9F) (resolution in the x direction was 0.53 and 0.32 mm, respectively with and without CF). The effect of the commercial arrays with the inter-array gap does introduce grating lobes that are apparent in FIG. 9B, with a height of ˜−80 dB after the introduction of CF weighting. Using CF, the side lobes were suppressed by 18 to 30 dB compared to the values without using CF (FIG. 9B).

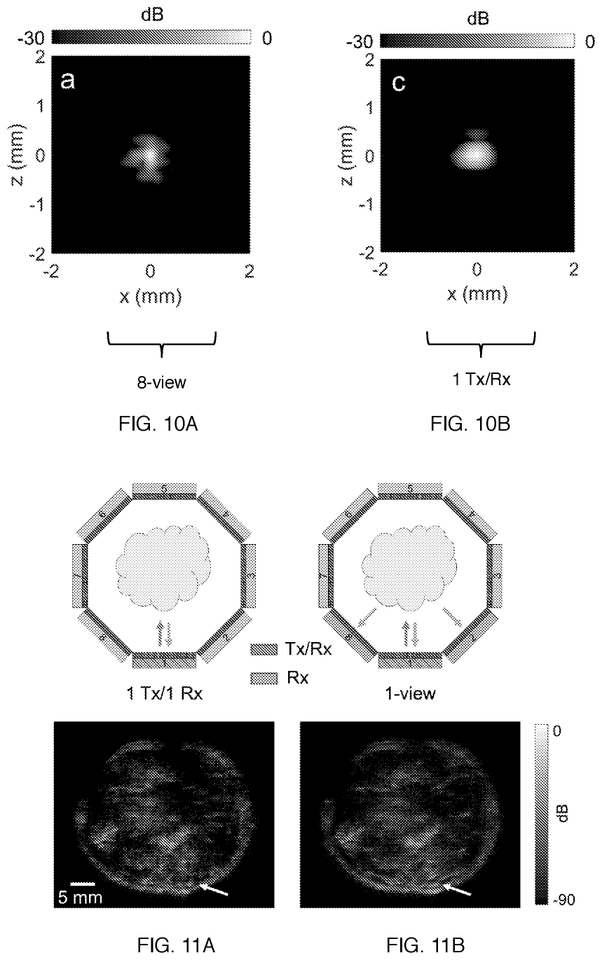

Experimental results were consistent with the simulation (FIGS. 10A-B). Using CF, the resolution in the x and z directions was 0.19 and 0.24 mm, respectively, using 8-view acquisition (FIGS. 10A-B), as compared to 0.43 and 0.32 mm, respectively for 1-array acquisition. Grating lobes introduced by the interelement gap had a height of ˜−60 dB with CF, suppressed by 22 dB as compared to the height (˜−38 dB) without using CF.

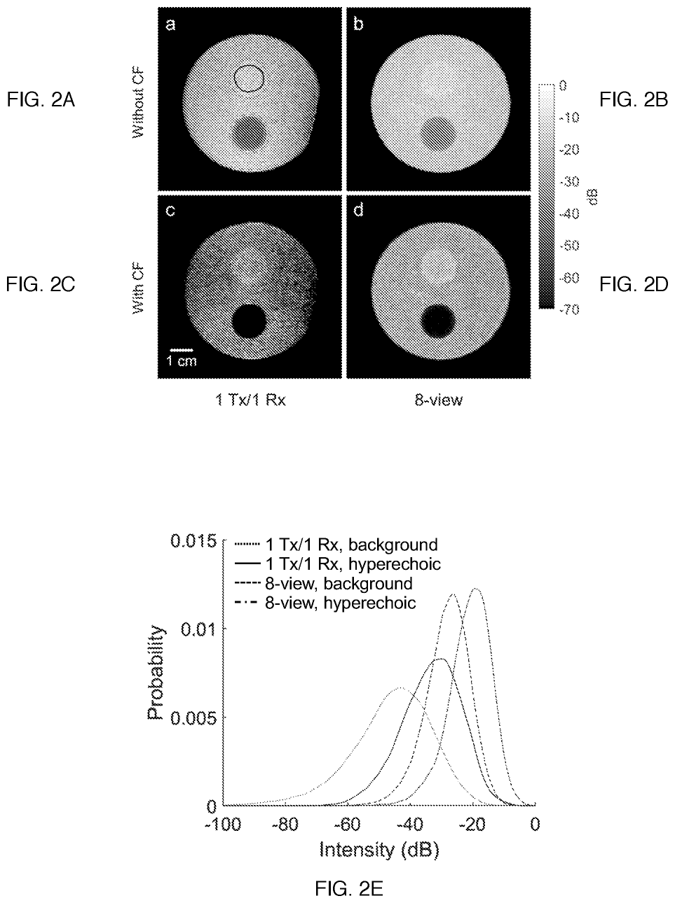

FIGS. 2A-E show B-mode images of a phantom containing hyperechoic and anechoic inclusions obtained from 1 Tx/1 Rx and 8-view acquisitions without CF (FIGS. 2A-B) and with CF (FIGS. 2C-D). The hyperechoic region is marked with a black circle as shown in FIG. 2A. Array 1 placed at the bottom of the images was used for the 1 Tx/1 Rx acquisition (FIG. 1F). sSNR, CR and CNR (see Tables 1 and 2 below) were evaluated using the envelope signals in the marked regions. FIG. 2E is a comparison of the probability density function of the intensity values in the background and hyperechoic regions for FIG. 2C and FIG. 2D.

Tomographic imaging with the full aperture and CF also improved image contrast (FIGS. 2A-E). The 8-view acquisition improved both the speckle signal-to-noise ratio (sSNR) and the contrast-to-noise ratio (CNR) compared to the acquisition with a single array. An increase of up to 53% (from 0.89 to 1.36) in sSNR was observed for the hyperechoic region, 2% (from 1.77 to 1.80) for the anechoic region, and 86% (from 0.65 to 1.21) in the background comparing the 8-view and 1 Tx/1 Rx acquisitions (Tables 1 and 2 below). The improvements in sSNR using 8-view acquisition are further illustrated by the comparison of the probability density function of intensity values in the background and hyperechoic regions (FIG. 2E). Acquisition with 8 arrays increases the mean intensity and reduces the standard deviation of the intensity in both the background and hyperechoic regions.

With the addition of CF weighting, the contrast ratio (CR) between hyperechoic and background regions and between anechoic and background regions increased by 2.2 dB (from 4.6 dB to 6.8 dB) and 30.4 dB (from −18.9 dB to −49.3 dB), respectively, for the 8-view acquisition (Tables 1 and 2). Comparing the 8-view and 1 Tx/1 Rx acquisitions, the CNR increased by 20% (from 0.55 to 0.66) between the hyperechoic and background regions, and by 85% (from −0.65 to −1.20) between anechoic and background regions (Table 2). The combinational use of CF and tomographic acquisition (FIG. 2D) improves visualization of the boundaries and reduces noise.

### A2b) Fine Anatomical Details are Revealed by Correction for SOS (Speed of Sound)

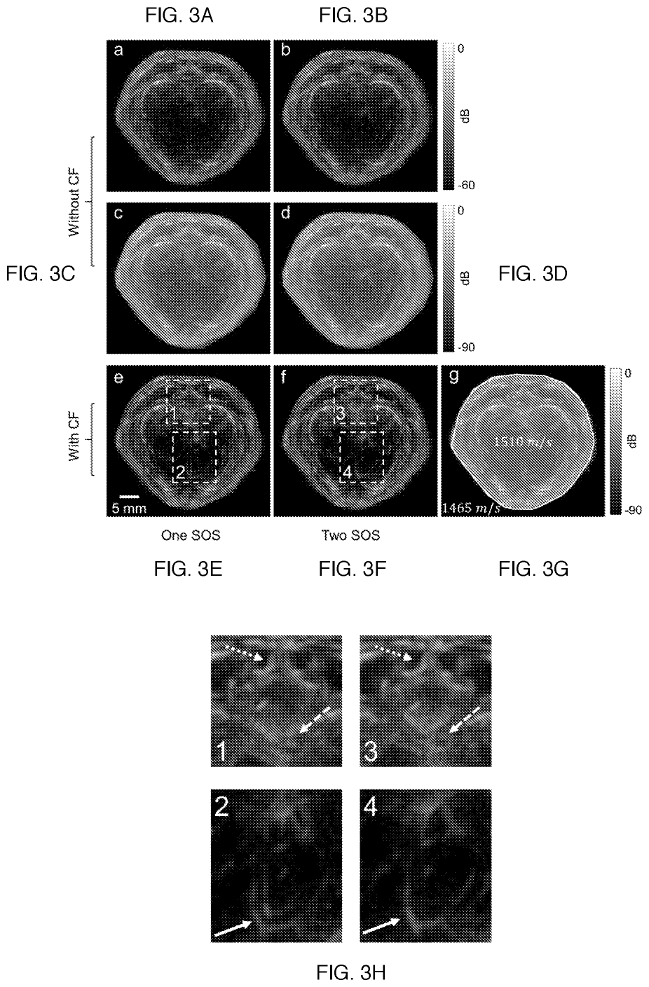

FIGS. 3A-F are a comparison of the cross-sectional image of rat chest reconstructed using single SOS without CF displayed with (FIG. 3A) 60 dB and (FIG. 3C) 90 dB dynamic range and with CF (FIG. 3E), and using two SOS without CF displayed with (FIG. 3B) 60 dB and (FIG. 3D) 90 dB and with CF (FIG. 3F). Images reconstructed using two SOS were achieved by correcting the delays for the pixels fall inside the segmented area (FIG. 3G) from the image reconstructed using a single SOS. FIG. 3H is a zoom over the outlined regions in (FIGS. 3E-F). Solid, dash and dot arrows indicate the location of the outer heart wall, the dorsal aorta and the vertebrae, respectively.

Applying CF weighting increases the dynamic range of the image which is evidenced by the comparison of the images with and without CF both displayed with 90 dB dynamic range (FIGS. 3C,E and FIGS. 3D,F). To facilitate the comparison between images with and without CF, a different (60 dB) dynamic range was used for the images without CF (FIGS. 3A-B) to ensure an almost equivalent histogram of pixel gray-scale levels in the images.

Consistent with the results in phantom studies, CF weighting improved the contrast of tomographic images of the rat abdomen (FIGS. 3A-H). In order to further improve tissue differentiation, reconstructions were created with correction for the speed of sound (SOS) inside tissue. Although many anatomical structures are distinguishable in the images reconstructed with a single SOS (FIGS. 3A,C,E), fine anatomical details were not easily visualized due to the delay estimation error inside the body. For instance, the vertebrae were enlarged, the location of the dorsal aorta was not obvious, and the heart wall boundaries were doubled (FIGS. 3E,H). Application of the dual SOS beamformer resolved these artifacts (FIGS. 3F,H). The results clearly reveal the capability of a dual SOS beamformer to provide high resolution anatomic imaging.

A2c) Image Fidelity is Improved with Large Imaging Coverage Angle

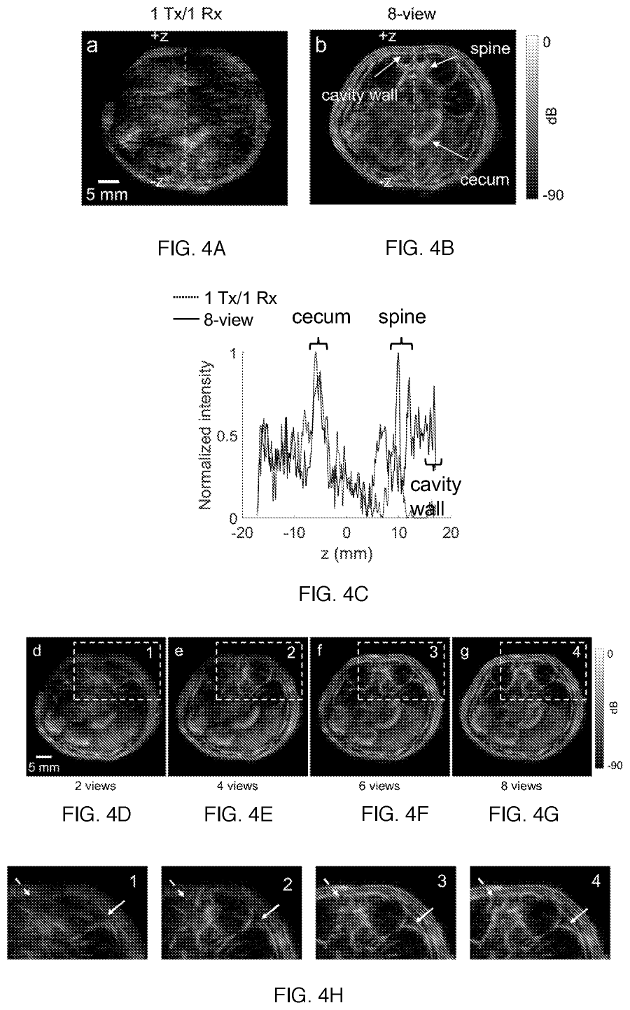

FIGS. 4A-C show a comparison of the cross-sectional image of a rat abdomen imaged with 1 Tx/1 Rx acquisition (FIG. 4A) and 8-view acquisition (FIG. 4B) and reconstructed with the dual SOS beamformer with CF. The dash lines in FIGS. 4A-B represent the location where the line profiles of FIG. 4C are extracted. FIGS. 4D-G show image fidelity is improved as the imaging coverage angle increases for acquiring (FIG. 4D) 2 views (view 1-2), (FIG. 4E) 4 views (view 1-4), (FIG. 4F) 6 views (view 1-6) and (FIG. 4G) 8 views (view 1-8), respectively. Views 1 to 8 are defined in FIG. 1F. FIG. 4H is a zoom over the outlined regions in (FIGS. 4D-G). The solid and dashed arrows indicate the left kidney and spine of the rat, respectively. See FIG. 1F for the definition of 1 Tx/1 Rx and 8-view acquisitions.

Applying the dual SOS beamformer, we evaluated the impact of the number of segments and views used in the tomographic reconstruction of the rat model. We tested whether the improvement in the tomographic view resulted from the use of three arrays vs one array on reception of a single view and found that resolution and field of view were improved by using three arrays in reception (FIGS. 11A-B, e.g. see the superficial layers indicated by the arrows). With the 8-view acquisition, the cross-section of rat abdomen was imaged and reconstructed with high fidelity (FIG. 4B). As a result, the spine, and the abdominal cavity, including the left kidney, cecum and small intestines, were clearly depicted (FIG. 4B). With 1 Tx/1 Rx acquisition, the reconstruction was incomplete, and the images lack fidelity due to poor image quality and the limited field of view (FIG. 4A). Direct comparison of the profiles (FIG. 4C) in the sagittal plane shows the improved discrimination of anatomical features with the tomographic reconstruction. We further demonstrate the effect of imaging coverage angle on image quality in FIGS. 4D-H. Acquiring 2 views, the left kidney of the rat was not visible, reconstruction of the dorsal abdominal wall was incomplete and only a portion of the spine was visible (FIGS. 4D, H). As the imaging coverage angle increases, more and more anatomical information was retrieved, and the image quality improved (FIGS. 4E-H). Finally, compounding all 8 views, the left kidney, spine and large intestines were clearly delineated (FIGS. 4G-H).

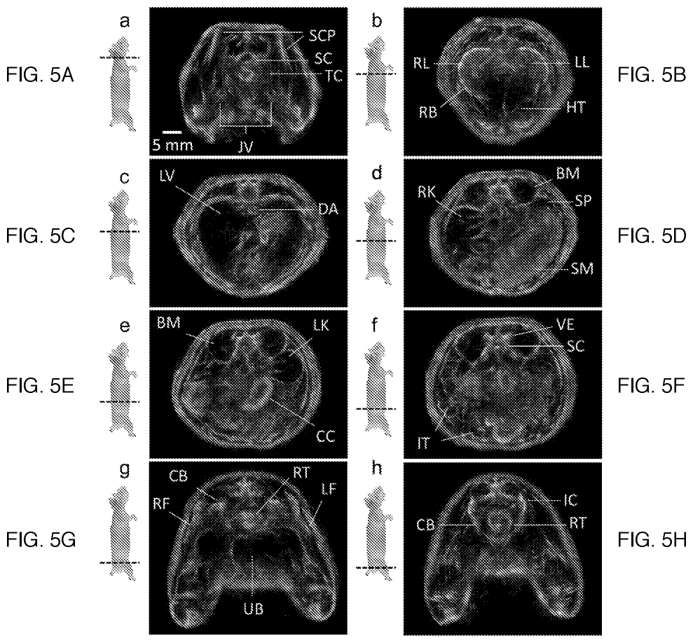

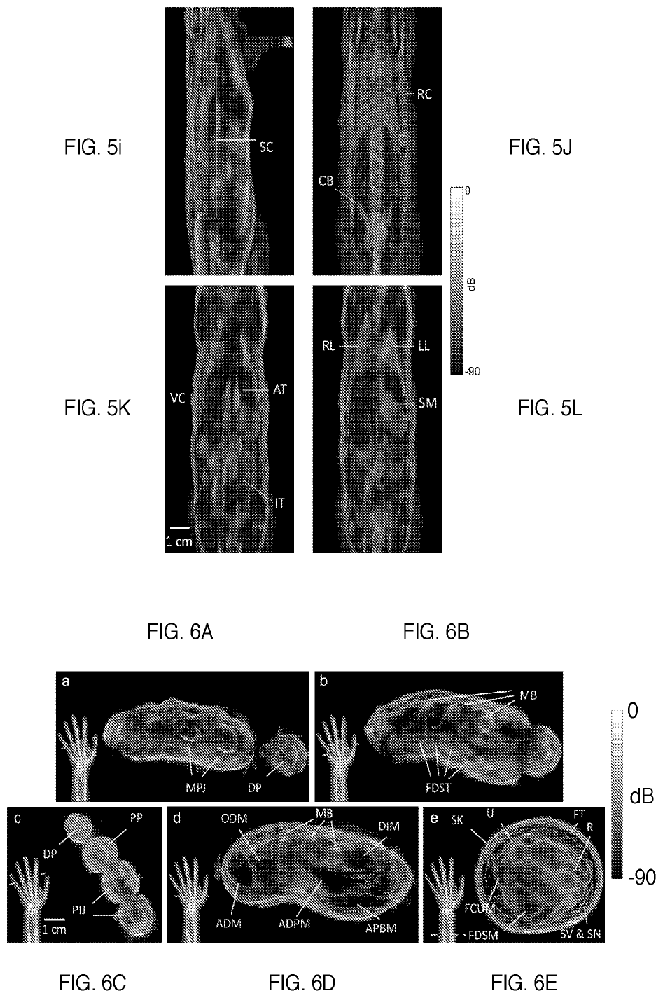

FIGS. 5A-L show ultrasound tomography of small-animal body anatomy. Cross-sectional images of the (FIG. 5A) upper and (FIG. 5B) lower thoracic cavity, (FIG. 5C) two lobes of the liver, (FIG. 5D) upper and (FIG. 5E) lower abdominal cavity, (FIG. 5F) abdominopelvic cavity, (FIG. 5G) upper and (FIG. 5H) lower pelvic cavity in the rat. FIG. 5i is a sagittal and FIGS. 5J-L are coronal views of the rat trunk. The figure legend is as follows: AT, aorta; BM, backbone muscles; CB, coxal bone; CC, cecum; CL, colon; DA, dorsal aorta; HT, heart; IC, Ischium; IT, intestines; LF, left femur; LK, left kidney; LL, left lung; LV, liver; JV, jugular vein; RB, rib; RC, rib cage; RF, right femur; RK, right kidney; RL, right lung; RT, rectum; SC, spine; SCP, scapula; SM, stomach; SP, spleen; TC, trachea; UB, urinary bladder; VC, vena cava; VE, vertebra.

Using 8 arrays, dual SOS beamformer and CF, high fidelity images of the anatomical structures in thoracic (FIGS. 5A-B), abdominal (FIGS. 5C-F) and pelvic cavity (FIGS. 5G-H) of the rat were successfully reconstructed. The thickest width of the trunk was ˜4.4 cm (FIGS. 5G-H). Most of the important anatomical features in all the cavities, including trachea, ribs, vertebrae, scapula, heart, livers, lungs, big vessels (jugular vein, dorsal aorta), kidneys, bladder, femur, rectum etc. are easy to identify and labeled in FIGS. 5A-L. Sagittal and coronal slices of the 3D reconstructed volume clearly present the entire spine cord (FIGS. 5i-J), rib cage (FIG. 5J), anatomical location of the lungs and stomach (FIG. 5K), vena cava, aorta and intestines (FIG. 5L).

FIGS. 6A-E show cross-sectional images of the human hand, wrist and forearm of a research subject. (FIG. 6A) proximal phalangeal joint section; (FIG. 6B) distal palm section; (FIG. 6C) proximal phalangeal section; (FIG. 6D) distal palm section; (FIG. 6E) upper wrist section. The figure legend is as follows: ADM, abductor digiti minimi muscle; ADPM, adductor pollicis muscle; APBM, abductor pollicis brevis muscle; DA, dorsal aponeurosis; DIM, dorsal interosseous muscle; DP, distal phalange; FCUM, flexor carpi ulnaris muscle; FDSM, flexor digitorum superficialis muscle; FDSM, flexor digitorum superficialis tendons; FT, fat; MB, metacarpal bone; MPJ, metacarpo-phalangeal joint; ODM, opponens digiti minimi muscle; PIJ, proximal interphalangeal joints; PP, proximal phalange; R, radius; SK, skin; SN, superficial nerve; SV, superficial vessel; U, ulna.

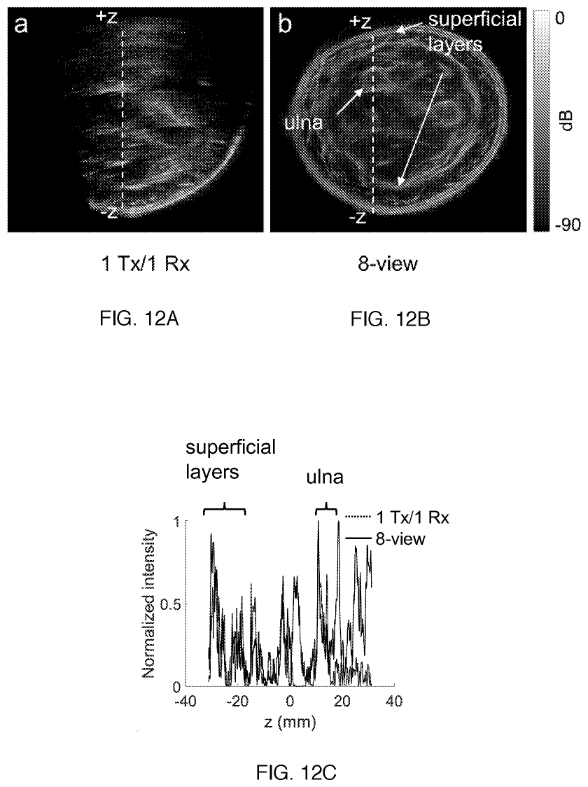

We also investigated the limited view effect in imaging of the human hand, wrist and forearm (FIGS. 12A-C) and the results are consistent with the comparison for rat imaging. With 1 array ˜50% of the cross-sectional area was visualized (FIG. 12A) and the radius and ulna were indistinguishable. With 8-view acquisition, the entire cross-section was reconstructed (FIG. 12B), the boundaries of the radius and ulna were well defined and tendons and superficial vasculature and nerves can be detected. The comparison of line profiles (FIG. 12C) clearly shows the enhanced view using 8 arrays. Cross-sectional images of the human hand in the phalangeal section (FIGS. 6A,C), palm (FIGS. 6B,D) and upper wrist were acquired (FIG. 6E). The largest dimension in the images in the hand imaged was ˜9.4 cm in the palm section (FIG. 6B). Anatomical structures including phalangeal bones, phalangeal joints, superficialis tendons and vessels, muscles, radius and ulna were identified (FIGS. 6A-E).

### A2d) Quantitative Mapping of Ultrasound Attenuation

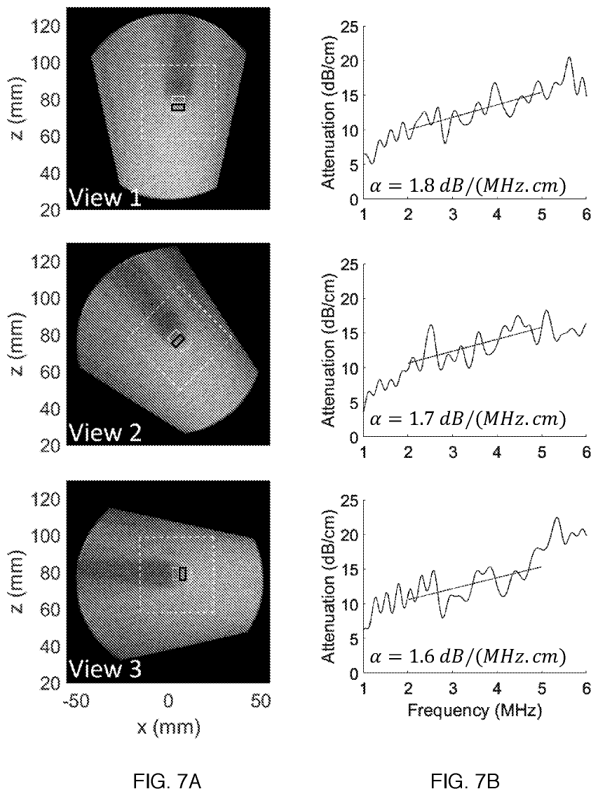

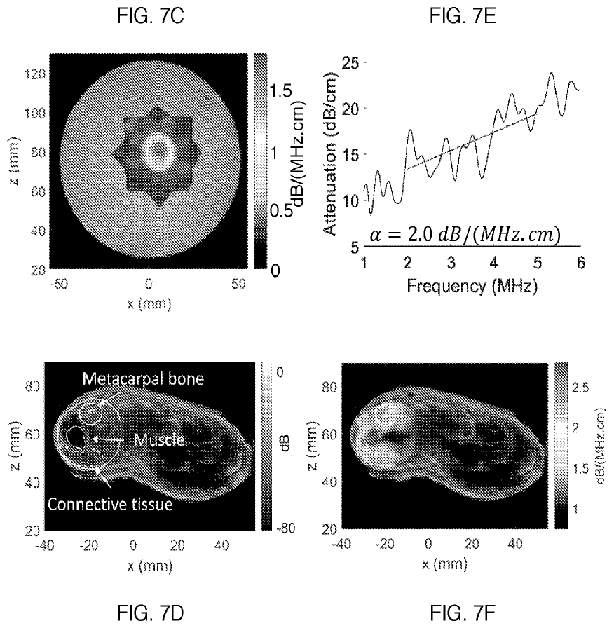

FIG. 7A shows B-mode images of a phantom containing an attenuating inclusion from different views. The white dashed boxes (27.5×27.5 mm²) outline the areas where ultrasound attenuation coefficients were estimated. The black and white solid boxes indicate the proximal and distal windows (with respective the Tx array) from which the spectral difference (FIG. 7B) was evaluated. The ultrasound attenuation coefficient (α) was estimated from the slope of the spectral difference between 2-5 MHz bandwidth. In FIG. 7C the local attenuation images from all the 8 views were compounded to produce the final attenuation coefficient image overlaid on B-mode image. The white circular contour outlines the boundary of the attenuation inclusion. FIG. 7D is a B-mode image of the transverse section of a human palm. The outer white contour outlines the region where the local ultrasound attenuation coefficients (FIG. 7E) were estimated to create the attenuation coefficient image for the human palm which is overlaid on B-mode image in FIG. 7F. The white solid, dot and dash contours indicate metacarpal bone, muscle, and connective tissue, respectively, which present distinct ultrasound attenuation coefficients as shown in FIG. 7F.

Finally, the tomographic system with CF and dual SOS provides the opportunity to quantify tissue properties in vivo. We therefore evaluated tomographic quantification of local attenuation as a demonstration of the feasibility of quantitative imaging with this system. The transition region where the image intensity decreases distal to the attenuating inclusion (creating a shadow artifact) was obvious in each view of the B-mode phantom images (FIG. 7A). Hence, a region-of-interest (ROI) of 27.5×27.5 mm² covering the inclusion was placed in the image (white dash boxes in FIG. 7A) where α was estimated locally to create an attenuation map for each view. A linear relationship between ultrasound attenuation and frequency was observed in the ROI (FIG. 7B). The attenuation maps reconstructed for all 8 views were then compounded to create a final attenuation image in the ROI (FIG. 7C) overlaid on the B-mode image. The attenuation image successfully localized the attenuation inclusion (FIG. 7C) with more than 1.3 dB/(MHz·cm) contrast to the background.

We further quantified the ultrasound attenuation coefficients in vivo in a transverse section of the human palm (FIGS. 7D-F). The attenuation image was created for a region containing the fifth metacarpal bone, muscle and connective tissue (FIGS. 7D,F). The ultrasound attenuation coefficient α (mean±std) was 1.33±0.17 dB/(MHz·cm), 1.67±0.21 dB/(MHz·cm), 2.05±0.17 dB/(MHz·cm) in the locations outlined for muscle, connective tissue and the metacarpal bone (FIG. 7F), respectively, showing the ability of our system to differentiate tissues with different ultrasound attenuation properties.

### A3) Discussion

We set out to develop a system for real-time quantitative reflection tomography and to apply this system for imaging phantom, small animal and human tissue. Programmable, high frame rate scanners with a high channel count provide an unprecedented opportunity to optimize tomographic acquisition for tissues for which through transmission is not optimal. Here, we first optimized acquisition using a subset of arrays for each view. The entire object was imaged by coherently compounding data from eight views. By combining a 3-array receive aperture with the acquisition of eight views, we achieved isotropic in plane resolution on the order of half wavelength. We found that the spatial resolution and contrast could be further improved by coherence factor weighting and by considering different speed of sound values within and outside the imaged object. We achieved a high-volume acquisition rate through the use of plane wave imaging, facilitating coherent compounded imaging and further increasing image quality. Taking advantage of ultrafast imaging with plane wave sequences and a large aperture in ultrasound tomography, anatomical information was acquired with sub-wavelength in-plane resolution in a large field of view in real-time.

Although our system can also be applied in transmission tomography mode, we use pulse-echo (reflection) signals from plane wave excitation in order to preserve real-time imaging. Consistent with previous work on improving image quality by increasing effective transducer aperture size, we also showed improvements in key image quality metrics including spatial resolution, sSNR, CR and CNR with tomographic imaging. Adding to the panoramic imaging capability, high resolution tomographic images in the rat were obtained. We further demonstrated the capability of our system by imaging the hand and wrist of a healthy volunteer. A fully tomographic view of anatomical features was obtained. These promising results show the potential of our system for orthopedic and myopathic imaging. With plane wave or diverging wave-based methods developed for Doppler, color flow, ultrasound attenuation and SOS imaging, and elastography, we foresee expanding ultrasound tomography to include these features.

The full aperture ring allows fast in-plane image acquisition within a few milliseconds with the 2-way travel of ultrasound waves ultimately limiting the acquisition rate. Here, tomographic imaging was carried out with an effective reconstruction frame rate of 10 Hz using a GPU. The imaging speed can be further improved using partial beamforming, i.e., beamform the images on each secondary system, then send the images to the primary system for final image formation. This scheme should improve the frame rate by at least a factor of 2 through relieving the computational burden and reducing the amount of data that needs to transfer between secondary and primary systems. With real-time imaging, tomographic functional imaging can be further developed.

In this work, we utilized a low-cost method of extending the transducer aperture using 3D printing. With this technology, assembly geometries can be designed to accommodate the requirements of a specific application and for an individual patient. However, one important limitation of this approach is that grating lobes caused by the missing transducer elements (in the gaps between the arrays) can degrade image quality, as has been shown in. A promising method for filling in missing data has been shown to be successful in geophysics using deep learning. For specific clinical applications, dedicated arrays will also be designed that will not involve gaps between elements. Section C below considers a different approach for gap compensation.

In addition, estimates of attenuation were achieved using tomographic plane wave acquisition. By combining images obtained from multiple acquisition directions, regions of locally enhanced attenuation were quickly recognized, and the attenuation coefficient estimated. Using the shadow-like artifact in the B-mode images from different views, high attenuation region in the phantom can be localized in real-time in a large field of view. The spectral-log-difference method applied here can apply a calibration procedure using a homogeneous phantom with known ultrasound attenuation coefficient to compensate for factors affecting the estimation, such as diffraction effects and backscattering. Nevertheless, without calibration, we were able to estimate the ultrasound attenuation coefficient of specific regions of interest and the estimated values reasonably differentiate tissues including the muscle, connective tissue and metacarpal bone in the human palm. To improve the quantitation, further work with a range of known materials is required.

### A4) Methods

### A4a) System Infrastructure

A real-time, programmable 1024-channel ultrasound platform was used to drive a customized tomographic transducer with 1024 elements. The primary system (Vantage 256, Verasonics Inc., Kirkland, Wash.) sends clock signals to synchronize three secondary Vantage 256 systems. A GPU card (RTX Titan, Nvidia, Santa Clara, USA) in the primary system speeds processing and the data transfer rate between the primary and each secondary system is 100 Gb/s. The tomographic transducer consists of 8 L7-4 linear arrays (ATL/Philips, Amsterdam, Netherlands) hosted in 8 3D printed sub-assembly pieces forming an octagonal manifold. Each array consists of 128 elements with a lateral pitch of 300 μm and an elevation width of 7.5 mm. The linear arrays were carefully positioned to align their respective elevation plane. The edge length of each sub-assembly (i.e. for 1 linear array) is about 6.2 cm, creating a large field of view of ˜13×13 cm². The lateral and axial directions with respect to array 1 were denoted as the x-direction and z-direction in the image, respectively (FIG. 9A).

A customized water tank having plastic membranes and 3D printed tank walls with acoustic windows opened to couple with the arrays was designed. Water was employed as the coupling agent between the arrays and the object.

### A4b) Imaging Sequence and Image Reconstruction

For one imaging view, 3 linear arrays (FIG. 1F) were activated and the center array consecutively transmitted several plane waves (5 MHz, 1 cycle pulse, no Tx apodization) of different angles between −13° and 13° with an even angle interval. The Tx array and its two neighboring arrays then received the reflected radio frequency (RF) signals (FIG. 1F) sampled at 20 MHz. For instance, when array 1 transmits plane waves, array 1, 2 and 8 receive the RF echoes and the image reconstructed using the data from these Tx/Rx events is denoted as view 1.

The image from one view (view j) was beamformed by delay-and-sum (DAS) method combined with coherence factor (CF) as,

rⱼ(x,z)=Σₙ₌₁^{N}Σₘ₌₁^{M}αₘₙsₘₙ(x,z(CFₘ(x,z), (1)

where n indexes the Rx channels and the total Rx channel number is N=384; M is the number of plane waves transmitted by each linear array and for the m^{th} Tx angle, sₘₙ(x,z) is the Hilbert Transform applied and delayed RF signal from each Rx channel; αₘₙ is the apodization coefficient which is set to 1 if the pixel position falls inside the propagation path of the plane wave and 25° acceptance angle of the Rx channel, or 0 otherwise; rⱼ is the beamformed image for view j; x,z are the spatial location of the image pixel and CF is expressed as,

$${CF}_{m} ( x , z ) = \frac{{\vert {\sum }_{n = 1}^{N} {s}_{m n} ( x , z ) \vert}^{2}}{N {\sum }_{n = 1}^{N} {\vert {s}_{m n} ( x , z ) \vert}^{2}} , \qquad ( 2 )$$

The active arrays were rotated to acquire images from 8 different views which were then coherently compounded to form the final tomographic images according to,

r(x,z)=Σⱼ₌₁⁸rⱼ(x,z), (3)

Then, the envelope of the beamformed data was detected and log compressed.

The secondary systems transfer the RF signals to the primary system for image reconstruction performed on the GPU card which allows real-time imaging capability. A frame rate of 10 frames/second was achieved for a 10×10 cm² image with an isotropic pixel size of one wavelength (0.3 mm) and 3 plane waves transmission per array setting, accounting for data transfer, processing and display.

As water is employed to couple the 8 arrays to the object, delay errors can occur in the presence of SOS mismatch between water and the object. Considering a single SOS to reconstruct the image, as commonly used in ultrasound imaging, would thus results in artifacts and degrades the image quality. For small animal and human hand imaging, we used a dual SOS beamformer following the procedure described hereafter.

We first reconstructed the image with single SOS. The image resolution can be set coarsely for a fast reconstruction, e.g. pixel size=1.2 mm. As the object was surrounded by water, its contour has a good contrast separation with the background and therefore can be selected manually or detected automatically using classic image processing methods, such as Grabcut. The image field was then partitioned to two domains with two SOS values. With the assumption that ultrasound rays travel straight from the source to the detectors, the paths along which the rays travel inside the subject were calculated to correct for the delay errors caused by the SOS difference in the object. With the corrected delays, the image was reconstructed with a finer pixel size of 0.15 mm.

As is shown in FIGS. 3A-H, with the dual SOS beamformer, the artifacts induced by SOS difference can be corrected to a large extent resulting much improved image quality.

### A4c) Image Quality Metrics

We used the following metrics to evaluate the image quality of our platform: 1) Spatial resolution, defined as the full-width-half-maximum (FWHM) of the PSF of the system; 2) Contrast ratio (CR)=20 log 10(μ₀/μ₁), where μ₀ and μ₁ are the mean values of the envelope signal in region 0 and 1, respectively; 3) Contrast-to-noise ratio (CNR)=(μ₀−μ₁)/\sqrt{σ₀²+σ₁²}, where σ₀ and σ₁ are the standard deviations of the envelope signal in region 0 and 1, respectively; and 4) Speckle signal-to-noise ratio (sSNR)=μ₀/σ₀, for region 0. Spatial resolution of our system was evaluated by imaging a point target at the center of the image filed in simulation and experiments (see section A4e for more details). CR, CNR, sSNR were evaluated by imaging an agarose-based tissue mimicking phantom fabricated following the procedure described in section A4f.

### A4d) Ultrasound Attenuation Imaging

Attenuation imaging was implemented using the spectral-log-difference method. Briefly, B-mode images from the 8 views were displayed in real-time. An ROI covering the high attenuating region was defined for each view based on shadowing distal to the inclusion in each direction (FIG. 7A). The ROI was then partitioned into different overlapping time-gated data blocks. Each data block was further partitioned to two non-overlapping windows (one distal and one proximal) of the same size (FIG. 7A) and the average power spectra, denoted as S_{(f,z)}, in the windows was estimated, where f is the frequency and z is the center depth of the window. The difference of the spectra between the two windows was calculated. Assuming linear dependency of ultrasound attenuation on frequency and omitting diffraction effects, the ultrasound attenuation coefficient α of each block was estimated according to,

log₁₀(S_{(f,z}_{ₚ}₎)/(S_{(f,z}_{_{d}}₎)=4(zₚ−z_{d})αf+R, (4)

where zₚ and z_{d} are the center depth of the proximal and distal windows, respectively; R is a constant related to the backscatter coefficients of the windows, assuming that the material in the windows has the same effective scatterer size. The block dimension was 7.5×7.5 mm² with a 90% overlap between successive estimates used in both the lateral and depth directions. Each block included 200 scanlines with 200 time samples in each scanline. The ultrasound attenuation coefficient was estimated from the slope of the spectral difference in the 2-5 MHz bandwidth. As the intensity in the proximal window is expected to be greater than that in the distal window and the high frequency components in the signals are expected to be more attenuated as compared with the low frequency components, two constraints were applied to remove invalid estimates. They were (1) α must be positive and (2) the spectral difference at 2 MHz estimated on the linear fitted slope must be positive. If the two conditions were not satisfied, the estimate for that data block was not retained. The ultrasound attenuation images from views 1 to 8 were compounded to create the final attenuation image which was then smoothed with a Gaussian filter of 0.3×0.3 mm² window size.

### A4e) Simulations

A point target was imaged in experiments and simulations to evaluate the spatial resolution of the system. The simulations were carried out using the Verasonics Research Ultrasound Simulator, with a point target of strong reflectivity defined at the center of the arrays. An ideal eight arrays geometry without gaps between arrays was first simulated and thereby the distance between the point target to the arrays was 4.6 cm (FIG. 1F). The exact transducer geometry as used in the experiments with inter-array gaps enforced was also simulated (FIG. 9A) and the distance between the point target and the arrays was 7.5 cm. The sequence depicted in FIG. 1F (8-view acquisition) with 3 plane waves transmitted per array (24 plane waves in total) was used to image the point target. For comparison, the same target was also imaged with a single array (FIG. 1F, 1 Tx/1 Rx) using 24 plane waves (−13° to 13°) to ensure that same amount of illumination was received. For the experiments, a metal wire (40 μm thickness) was vertically suspended in the water tank filled with degassed water and imaged using the same sequences. PSFs of the system were extracted from the B-mode images of the point target and the spatial resolution was determined. Nylon wire grid targets (50 μm thickness) were also vertically suspended in the water tank filled with degassed water and imaged using the tomographic acquisition with 1, 3 and 5 arrays used in reception (FIGS. 8A-i).

### A4f) Phantom Synthesis

Image quality metrics including sSNR, CR and CNR were evaluated on an agarose-based tissue-mimicking phantom with two cylindrical inclusions (one hyperechoic and one anechoic). The background substrate of the phantom was prepared by dissolving 1.5% (w/v) agarose powder (A10752, Alfa Aesar) in degassed water at 80° C. and mixed with 1% (w/v) silicon carbide (SiC) powder (A16601, Alfa Aesar) homogeneously using a magnetic stirrer (SH-2, Faithful) prior to solidification. The hyperechoic inclusion (1.5% agar w/v, 2% SiC w/v) was prepared following the same procedure as for preparing the background substrate. The anechoic inclusion was filled with water (FIGS. 2A-E). Comparison was made following the same settings for the point target experiments described above.

An agarose-based (1.5% w/v agar, 0.5% w/v SiC) tissue mimicking phantom with one high ultrasound attenuation inclusion in the center was prepared following the same procedure described above. The inclusion was obtained by adding an additional 13% w/v aluminum oxide powder (#3 Micron, Beta Diamond Products) to the agarose solution. The ultrasound attenuation coefficient of the inclusion and the background substrate measured by insertion loss techniques were 2.3 and 0.1 dB/(MHz·cm), respectively. We then imaged this inclusion phantom with 56 plane waves (8-view acquisition, 7 plane waves per array, −13° to 13).

### A4g) Small Animal and Human Imaging Protocols

All animal experimental procedures were performed in accordance with protocols approved by the local Institutional Animal Care and Use Committee. A 7-week old female rat (200 g body weight) was anesthetized with vaporized isoflurane (1 L/min of oxygen and 2% isoflurane) gas system and then, the hair was removed using clippers and depilatory cream. The rat was humanely euthanized and placed in the water tank for imaging. The rat was secured to a 3D printed mold holding its head and weight was applied to the tail to ensure an upright position during imaging. For 3D tomographic imaging, the rat was mounted on two linear translation stages which were motorized by a motion controller (ESP 300, Newport, Irvine, Calif., USA). The region between the base of the neck to the base of the tail was scanned in the transverse plane with 1 mm interval between scans. In total, 130 slices were acquired (covering 130 mm) and 56 plane waves (8-view acquisition, 7 plane waves per array, −13° to 13) were used for imaging each slice.

A healthy female volunteer (31 years old) was recruited for the in vivo tomographic imaging of the forearm, wrist and hand. All imaging procedures followed the protocol approved by local Review Board. Informed consents were received from the volunteer after explaining the protocol. Imaging was performed with the same sequence used for the small animal scan described in the previous paragraph. During the experiments, the volunteer was instructed to immerse the left hand and forearm vertically in the water tank. Tomographic images were acquired on the fly while the volunteer moved the hand and forearm freely.

### A5) Supplementary Material

To calibrate the location of the transducer elements, each L7-4 array transmitted a plane wave of 0° steering angle to insonify two static targets (40 μm wires spaced by ˜15 mm) and recorded the backscattered echoes. The targets were immersed in water, placed at the center of the field-of-view and oriented to avoid overlap between the echo traces. The delay associated to each target was estimated with cross-correlation between the channels. The relative position (x, z) of the two targets with respect to the transmitting array was then recovered by fitting the delay trace associated with the target position (x, z) to the measured delays. The water speed-of-sound was set based on the measured temperature. After determining the target's location with respect to each of the 8 arrays, translation and rotation were applied to each array location to obtain their absolute position with respect to array 1.

FIGS. 8A-i show simulation of the point spread function (PSF) for the system and the nylon wire targets (50 μm thickness) imaged by the commercial arrays with the inter-array gap for 8-view acquisition with (FIGS. 8A-C) 1 Tx/1 Rx, (FIGS. 8D-F) 1 Tx/3 Rx and (FIGS. 8G-i) 1 Tx/5 Rx for each view. FIGS. 8B,E,H are simulated B-mode images of a point target located at the center of the arrays with coherence factor weighting. FIGS. 8C,F,i are B-mode images of the nylon wire targets with coherence factor weighting. FIG. 8J show the PSFs along x-direction for the simulated point target. FIG. 8K shows RF data of the nylon wire targets recorded by array 1-3 with array 1 as the transmitting array.

FIGS. 9A-F show simulation of the PSF for acquisition with (FIG. 9A) the commercial arrays with the interelement gap. FIG. 9B shows the PSFs along x-direction for 8-view and 1 Tx/1 Rx acquisitions, with or without coherence factor weighting. (c-f) For 8-view acquisition, simulated B-mode images of a point target located at the center of the arrays with or without coherence factor weighting are shown on FIGS. 9C-D. The arrays are positioned along a 15 cm diameter circle, i.e. the distance between array 1 and 5 is 15 cm. For 1 Tx/1 Rx acquisition, simulated B-mode images of a point target located at the center with or without coherence factor weighting are shown on FIGS. 9E-F. Same sequence as used for the simulations in FIGS. 1F-J was used for the simulations here.

A metal wire (40 μm thickness) suspended vertically at the center of the commercial arrays with the inter-array gap was imaged to show the PSFs. FIGS. 10A-B show simulated B-mode images for 8-view and 1 Tx/Rx, respectively. Same sequence as used for the simulations in FIGS. 1F-J was used for the experiments here.

TABLE 1
Without CF, summary of sSNR, CR and CNR in the
hyperechoic, anechoic and background regions
imaged with 1 Tx/1 Rx and 8-view acquisitions.

| | sSNR | | CR (dB) | | CNR | |
|---|---|---|---|---|---|---|
| | 1 Tx/1 | 8- | 1 Tx/1 | 8- | 1 Tx/1 | 8- |
| | Rx | view | Rx | view | Rx | view |
| Hyperechoic | 1.80 | 1.87 | 5.8 | 4.6 | 0.69 | 0.66 |
| Anechoic | 1.77 | 1.80 | −24.5 | −18.9 | −1.10 | −1.58 |
| Background | 1.18 | 1.79 | — | — | — | — |

TABLE 2
With CF, summary of sSNR, CR and CNR in the hyperechoic,
anechoic and background regions imaged with
1 Tx/1 Rx and 8-view acquisitions.

| | sSNR | | CR (dB) | | CNR | |
|---|---|---|---|---|---|---|
| | 1 Tx/1 | 8- | 1 Tx/1 | 8- | 1 Tx/1 | 8- |
| | Rx | view | Rx | view | Rx | view |
| Hyperechoic | 0.89 | 1.36 | 9.7 | 6.8 | 0.55 | 0.66 |
| Anechoic | 1.11 | 1.09 | −53.9 | −49.3 | −0.65 | −1.20 |
| Background | 0.65 | 1.21 | — | — | — | — |

FIGS. 11A-D show a comparison of the cross-sectional images of the rat abdominal cavity from (FIG. 11A) 1 Tx/1 Rx acquisition and (FIG. 11B) 1-view acquisition (1 Tx/3 Rx) reconstructed with coherence factor weighting. The arrows indicate the boundaries of anterior superficial layers showing improved spatial resolution and image quality by just increasing the receiving transducer aperture size in the 1-view acquisition.

FIGS. 12A-C shows a comparison of the cross-sectional images of human wrist with 1 Tx/1 Rx acquisition (FIG. 12A) and 8-view acquisition (FIG. 12B) and reconstructed with the dual SOS beamformer without and with CF. The dash lines in FIGS. 12A-B represent the location where the line profiles of FIG. 12C are extracted.

### B) Dual Modality

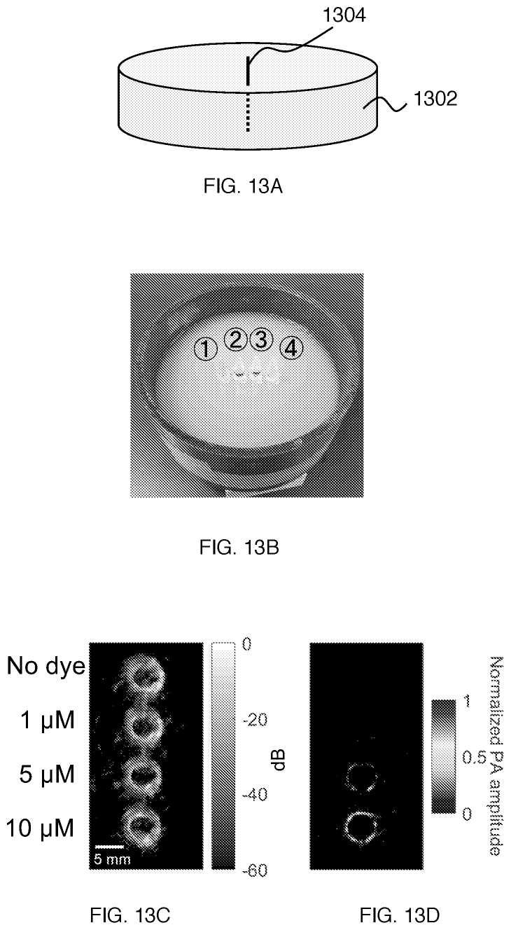

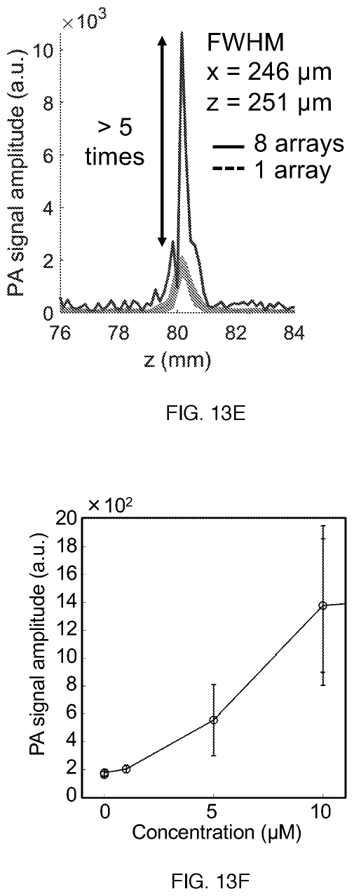

The present approach can be used in dual-modality configurations, where any compatible imaging modality is combined with improved acoustic imaging as described above. FIGS. 13A-F show one such example, where the second imaging modality is photoacoustic imaging. In photoacoustic imaging, light is absorbed by the target and local heating etc. leads to the production of acoustic waves which are then imaged by the system. Here FIG. 13A shows a phantom 1302 (e.g., 1.5% agar +0.5% intralipid) including a 40 μm diameter wire 1304. FIG. 13B shows four inclusions in phantom 1302 having various concentrations of ICG (indocyanine green) dye. Inclusion 4 has no ICG and the concentrations of ICG in inclusions 1, 2, and 3 are 10 μM, 5 μM, and 1 μM, respectively.

FIG. 13C is the acoustic image of the phantom and FIG. 13D is the corresponding and co-registered photoacoustic image of the phantom. It is apparent that at higher levels of dye concentration a photoacoustic image of the inclusion in the phantom is formed that is aligned with the corresponding acoustic image. FIG. 13E shows that use of 8 arrays is substantially better than use of only one array for the photoacoustic imaging. FIG. 13F shows the dependence of photoacoustic signal amplitude on dye concentration for this example.

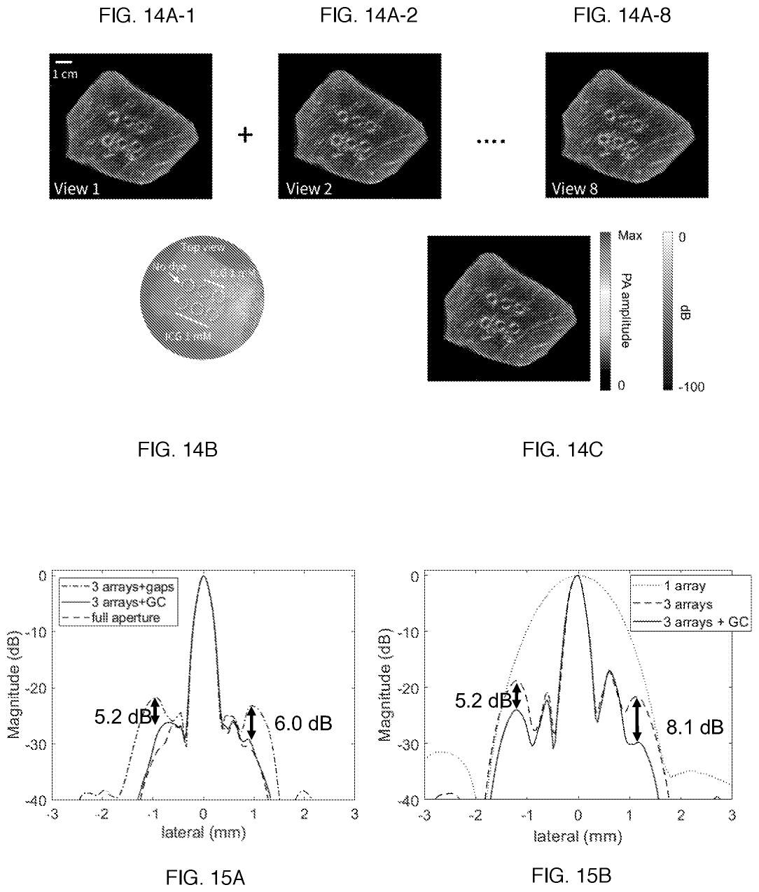

FIGS. 14A-1, 14A-2, 14A-8, 14B and 14C show another example of such dual modality imaging. Here FIGS. 14A-1, 14A-2, 14A-8 show individual images from views 1, 2, and 8. FIG. 14C is the result of properly combining the 8 views. FIG. 14B shows the phantom configuration for this experiment. Here the inclusions in the phantom are tubes having ICG solution in them as shown, covered by −1.5 cm thick chicken breast muscle.

### C) Gap Compensation

High throughput ultrasound systems allowing full control over a high number of channels (>256) are emerging as a powerful tool to improve imaging. This new technology development driven by volumetric imaging enables real-time control of a high number of elements. For 1D-array geometries this translates to the use of large apertures which improves image quality and offers a wider field-of-view suited for whole organ imaging.

In this section, we explore such configurations by combining three commercial phased arrays and test the improvements achieved by a large aperture with 384 elements and 10 cm aperture. Employing phased arrays, with a wide acceptance angle, offers an efficient way to interrogate the tissue with a limited number of transmit events using diverging waves (DW). Moreover, an auto-regressive filter is applied on virtual receive elements filling the inter-array gaps to mitigate the associated grating lobes.

The multi-array assembly is composed of three P6-3 (ATL/Philips, Amsterdam, Netherlands), each having 128 elements (0.22 mm pitch). The arrays are held together by a stackable 3D-printed manifold designed in-house. The aperture extends over 98.5 mm laterally with inter-array gaps of 9.3 mm resulting in visible grating lobes on bright reflectors. The arrays are interfaced to 2 Vantage 256 system (Verasonics, Kirkland, USA) which are part of the volume imaging package and allowing real-time control and processing. The position of the arrays was first calibrated with wire targets in water. Imaging was then performed at 4.5 MHz using diverging waves. Delay-and-sum beamforming was implemented on a GPU (Titan RTX, Nvidia, Santa Clara, USA) for real-time imaging.

To reduce the grating lobes generated by the physical gaps, 84 virtual receiving elements were created on receive and their associated signals estimated with an auto-regressive filter (FIG. 1E). In this approach, the frequency-domain signal at the (n+1)th channel, S_{f}(n+1) can be expressed as a linear combination of the signals at the p preceding channels as:

S_{f}(n+1)=α_{f}(1)S_{f}(n)+α_{f}(2)S_{f}(n−1)+ . . . +α_{f}(p)S_{f}(n−p+1)

with a_{f} being the coefficients of the filter. In this work, we chose p=8. The coefficients a_{f} are first estimated over the bandwidth of the transducer from the time delayed radiofrequency signals. Then the filter is used to predict signals on the virtual elements.

The improvements in term of lateral resolution at different depths were investigated with wire targets (40 μm diameter) placed at different depth and immersed in degassed water (FIGS. 15A-B). FIGS. 15A-B show lateral cross section of the point spread function obtained from a wire target located at a depth of 100 mm. FIG. 15A is a comparison between the PSF obtained with 1 array, 3 arrays (with gaps) and 3 arrays with GC. FIG. 15B shows a simulated PSF for 3 arrays (with gaps), 3 arrays with GC and an equivalent fully populated aperture.

The lateral resolution was calculated from the experimental point spread function as the full width half maximum (Table 3). The lateral resolution was determined to improve by a factor of 3 at a depth of 50 mm and a factor of 2 at a depth of 125 mm. At 50 mm and 125 mm, the equivalent f-number of the multi-array aperture is 0.5 (1.8 with 1 array) and 1.25 (4.5 with 1 array) respectively.

As expected, the inter-array gaps induced grating lobes which can be seen in the PSF cross-sections in FIGS. 15A-B. The grating lobes can be effectively reduced with the gap compensation method (FIG. 15A) and we measured experimental reductions ranging from 5 to 9 dB. To further evaluate the ability of the gap compensation (GC) method to reduce grating lobes, we performed simulations of the array geometry imaging a wire target (FIG. 15B). The simulation results show that the auto-regressive filter can recover the missing spatial information and produce a PSF almost similar to a fully populated aperture without gaps. In this ideal case the reduction is on the order of 6 dB.

TABLE 3
Lateral resolution measured as the full-width half-
maximum of the experimental point spread function.

| | FWHM (mm) | |
|---|---|---|
| Depth (mm) | 1 array | 3 arrays |
| 50 | 0.75 | 0.25 |
| 75 | 0.95 | 0.40 |
| 100 | 1.20 | 0.75 |
| 125 | 1.75 | 0.95 |

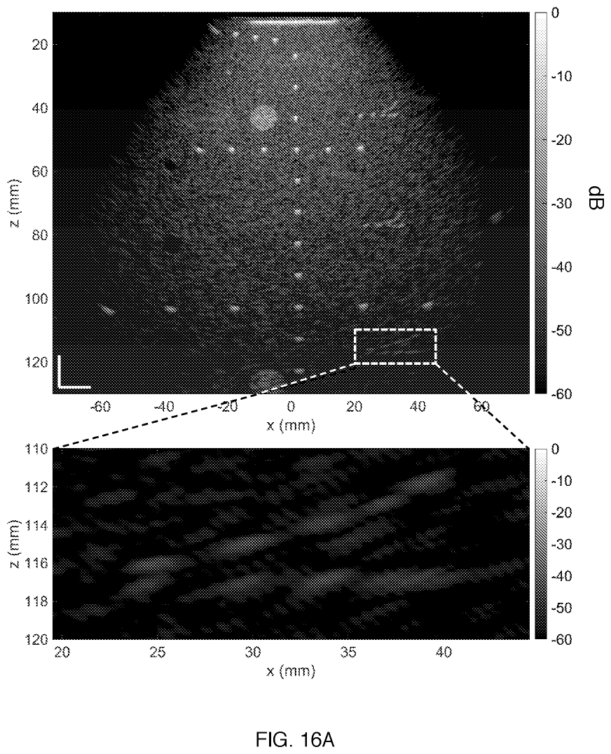

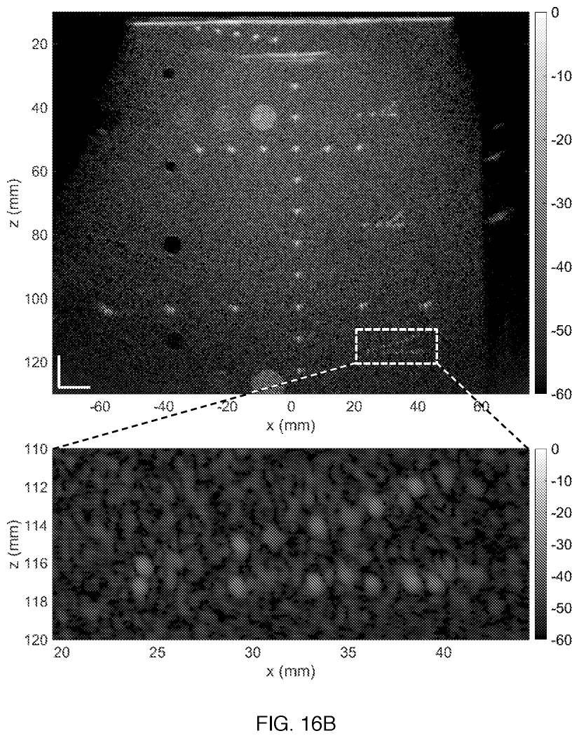

Imaging was performed on a multi-purpose ultrasound phantom containing various targets (model 040GSE, CIRS, USA; speed of sound 1540 m/s, attenuation 0.5 dB/MHz/cm). 30 DW were used for imaging with either the central array or all 3 arrays (10 DW per array) (FIGS. 16A-B). The improvement in field-of-view and resolution are visible throughout the image, and particularly demonstrated for deeper targets.

FIGS. 16A-B show imaging of a calibrated phantom. FIG. 16A shows an image reconstructed only with the central array (30 DW) and FIG. 16B shows an image reconstructed with all 3 arrays (10 DW per array, 30 DW total). The dynamic range is 60 dB. The zoomed sections display the wire targets located at a depth of 115 mm.

The array assembly was tested on a healthy volunteer (34 years old) following the local protocol approved by the local Institutional Review Board. Informed consent was received from the volunteer after explaining the protocol. The array was positioned under the ribcage to image the liver. Acquisition was performed on the fly during the scan (real-time beamforming). For comparison, both the 1 array (30 DW) and 3-array (also 30 DW) sequences were acquired at the same time.

The multi-array acquisitions show here more defined structures (mostly vessels) compared to the single array acquisitions.

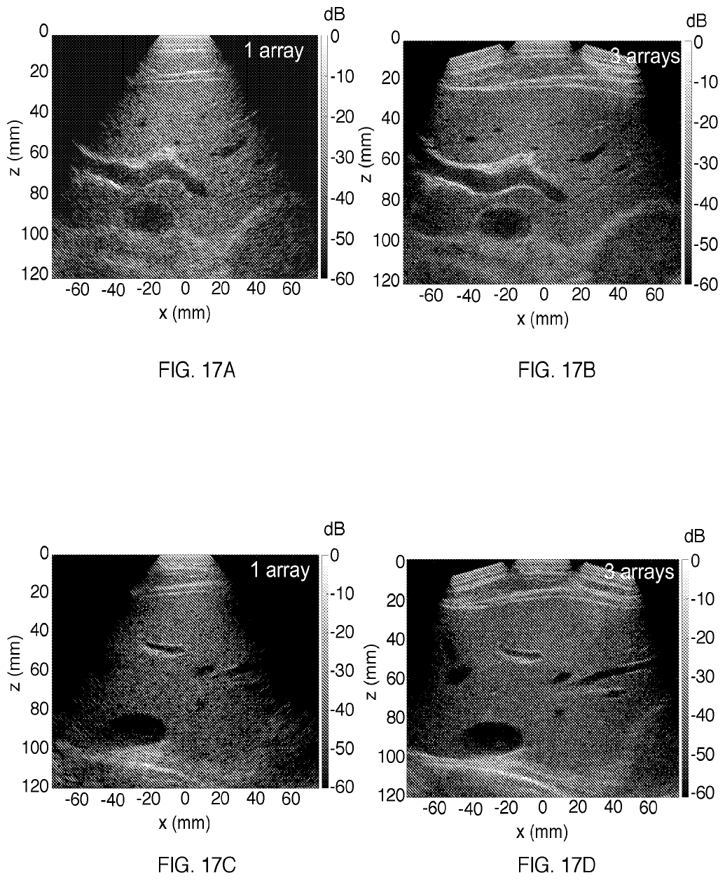

FIGS. 17A-D show in vivo liver imaging of a healthy volunteer. FIGS. 17A,C show images reconstructed with only the central array (30 DW total) and FIGS. 17B,D show images reconstructed with all 3 arrays (10 DW per array, 30 DW total). The dynamic range is 60 dB. The image size is 120×150 mm (axial×lateral).

The array assembly presented in this work allowed imaging of a wide field of view with improved resolution as demonstrated both in vitro and in vivo. Evaluation of the point-spread function on wire targets showed a lateral resolution improvement of a factor of 2 and above compared to using a single array. The use of virtual elements on receive for which signals are predicted with an auto-regressive filter yielded a significant reduction of the gap-related grating lobes. This modular configuration facilitates imaging of entire organs with improved metrics which is here particularly noticeable for deeper targets as seen on the commercial phantom. Initial evaluation of the multi-array configuration on the liver showed enhanced diagnostic capabilities.

1. Apparatus for performing acoustic tomography, the apparatus comprising:
three or more transducer modules disposed to surround a target;
wherein each transducer module includes two or more individually driven acoustic transducer elements;
a processor configured to transmit one or more first acoustic signals from a selected transducer module and configured to receive one or more second acoustic signals at two or more of the transducer modules, wherein the two or more of the transducer modules includes the selected transducer module;
wherein the one or more first acoustic signals are plane wave excitations or diverging wave excitations provided by the selected transducer module;
wherein the processor is further configured to sequentially select each of the transducer modules as the selected transducer module to provide a data set for tomographic reconstruction;
wherein the processor is configured to provide a first image from the data set for tomographic reconstruction.

2. The apparatus of claim 1, wherein the processor is configured to provide a frame rate for the first image of 10 Hz or more.

3. The apparatus of claim 1, wherein the plane wave excitations are provided by driving the acoustic transducer elements of the selected transducer module in phase with each other.

4. The apparatus of claim 1, wherein the plane wave excitations are provided by driving the acoustic transducer elements of the selected transducer module with a linear phase gradient to provide beam steering.

5. The apparatus of claim 1, wherein the diverging wave excitations are provided by defining a virtual source and driving the acoustic transducer elements of the selected transducer module with phases corresponding to the virtual source.

6. The apparatus of claim 5, wherein steering the diverging wave excitations is provided by disposing the virtual source at a corresponding location.

7. The apparatus of claim 1, wherein the two or more of the transducer modules does not include all of the transducer modules.

8. The apparatus of claim 1, wherein the data set for tomographic reconstruction is a reflection tomography data set.

9. The apparatus of claim 1, wherein the data set for tomographic reconstruction is a transmission tomography data set.

10. The apparatus of claim 1, wherein the processor is configured to apply a coherence factor correction to the data set for tomographic reconstruction.

11. The apparatus of claim 1, wherein the first image is a B-mode image.

12. The apparatus of claim 1, wherein the first image is an attenuation image.

13. The apparatus of claim 1, wherein the processor is configured to determine a speed of sound correction by estimating a target speed of sound in the target and an ambient speed of sound in a medium surrounding the target and between the target and the three or more transducer modules.

14. The apparatus of claim 1, wherein the apparatus is configured to provide a second image according to a second imaging modality that is co-registered with the first image.

15. The apparatus of claim 14, wherein the second imaging modality is photoacoustic imaging, and further comprising:
an optical source configured to provide an optical signal to the target;
wherein the two or more of the transducer modules further receive one or more third acoustic signals due to a photoacoustic effect in the target;
wherein the second image is a photoacoustic image determined from the third acoustic signals.

16. The apparatus of claim 1, wherein the processor is configured to reduce an effect of physical gaps between the transducer modules by:
defining two or more virtual acoustic elements at locations between the transducer modules and
estimating received acoustic signals at locations of the virtual acoustic elements.
